(19) United States
(12) Patent Application Publication
(10) Pub. No.: US 2014/0086864 A1
(43) Pub. Date: Mar. 27, 2014

(54) Shampoo Compostion

(71) Applicants: Tsunayuki Ishimori, Yokohama-shi (JP); Takahiro Yamashita, Yokohama-shi (JP)

(72) Inventors: Tsunayuki Ishimori, Yokohama-shi (JP); Takahiro Yamashita, Yokohama-shi (JP)

(73) Assignee: SHISEIDO COMPANY, LTD., Chuo-ku, Tokyo (JP)

(21) Appl. No.: 14/117,660

(22) PCT Filed: May 23, 2012
(86) PCT No.: PCT/JP2012/063166

§ 371 Date: Nov. 14, 2013

(30) Foreign Application Priority Data
May 25, 2011 (JP) 2011-116992

(51) Int. Cl. A61K 8/87; A61Q 5/12; A61K 8/81; A61Q 5/02; A61K 8/46; A61K 8/42

(52) U.S. Cl. CPC A61K 8/87; A61K 8/466; A61K 8/42; A61K 8/8158; A61Q 5/02; A61Q 5/12
USPC 424/70.122

(57) ABSTRACT

A shampoo composition excellent in fading-inhibiting effect and use feeling is provided. The shampoo composition is characterized by comprising:
(i) an anionic surfactant which is a taurine-derivative surfactant;
(ii) an amphoteric surfactant which is an alkylamide betaine surfactant;
(iii) a cationic conditioning polymer; and
(iv) 0.01 to 1 mass % of a quaternary ammonium group-containing silylated urethane polymer.

### RELATED APPLICATIONS

This application claims the priority of Japanese Patent Application No. 2011-116992 filed on May 25, 2011, which are incorporated herein by reference.

### FIELD OF THE INVENTION

The present invention relates to a shampoo composition, particularly to improvement in the hair color fading-inhibiting effect and the use feeling thereof.

### BACKGROUND OF THE INVENTION

In recent years, the coloring of the hair using an oxidation dye has widely been performed. The main troubles of people who enjoy the coloring of the hair include poor color durability, remarkable fading usually in about one month after dyeing, and inability to maintain beautiful hair color without frequently repeating dyeing. It is known that the fading of the oxidation dye is caused by the penetration of water into the inside of the hair when the hair is washed or the like to thereby wash away the dye incorporated in the inside of the hair, and thus a shampoo, a conditioner, or the like having a hair color fading-inhibiting effect is required.

Patent Literature 1 (Japanese Unexamined Patent Application Publication No. 2004-315369) describes a shampoo having a fading-inhibiting effect in which a silylated peptide-silane compound copolymer composition is blended. Further, Patent Literature 2 (Japanese Unexamined Patent Application Publication No. 2003-176214) describes a hair treatment having a fading-inhibiting effect containing a lipophilic cationic surfactant and a sterol. However, all of them have an insufficient effect and failed to provide an actual feeling of the durability of hair color.

### DISCLOSURE OF THE INVENTION

### Problem to be Solved by the Invention

Further, in the case of hair coloring using an oxidation dye, some coarseness and stiffness may occur after dyeing, due to the damage of the hair accompanying decolorization. Therefore, in the case of the hair after dyeing, the difference in the finger-running-through-hair properties and pliability of the hair when rinsing a shampoo tends to be felt stronger than in the case of the conventional hair. Therefore, it has been required not only to blend a component having high fading-inhibiting effect with a shampoo to be used for the colored hair but also to have a component more excellent in use feeling such as finger-running-through-hair properties and pliability than those to be blended in a conventional shampoo.

However, the construction of the components which can enhance the effect of the blended component having a fading-inhibiting effect and are significantly excellent in use feeling has not been obtained at present.

The present invention has been made in light of the above problems, and an object of the present invention is to provide a shampoo composition excellent in fading-inhibiting effect and use feeling.

### Means to Solve the Problem

As a result of intensive studies to solve the above problems, the present inventors have found that a quaternary ammonium group-containing silylated urethane polymer greatly inhibits the fading of the hair color after a shampoo, and a further improvement in the fading-inhibiting effect and excellent use feeling in the hair after dyeing are given by using a specific anionic surfactant, an amphoteric surfactant, and a cationic conditioning polymer in combination with the above component. The present invention has been completed on the basis of these findings.

Thus, a shampoo composition of the present invention is characterized by comprising:

(i) an anionic surfactant which is a taurine-derivative surfactant;

(ii) an amphoteric surfactant which is an alkylamide betaine surfactant;

(iii) a cationic conditioning polymer; and

(iv) 0.01 to 1 mass % of a quaternary ammonium group-containing silylated urethane polymer.

Also, in the shampoo composition, it is preferred that the (iii) cationic conditioning polymer comprises one or more selected from a trimethylaminopropylacrylamide chloride/dimethylacrylamide copolymer and an acrylic acid/methyl acrylate/methacrylamide propyltrimethylammonium chloride copolymer.

Also, in the shampoo composition, it is preferred that an amount of the (i) anionic surfactant blended is 1 to 20 mass %, and an amount of the (ii) amphoteric surfactant blended is 1 to 20 mass %.

Also, in the shampoo composition, it is preferred that an amount of the (iii) cationic conditioning polymer blended is 0.01 to 2 mass %.

### Effect of the Invention

The present invention can provide a shampoo composition excellent in fading-inhibiting effect on the hair after dyeing and use feeling such as finger-running-through-hair properties and pliability during rinsing.

### DESCRIPTION OF THE PREFERRED EMBODIMENT

Hereinafter, preferred embodiments of the present invention will be described in detail.

The shampoo composition according to the present invention contains (i) an anionic surfactant, (ii) an amphoteric surfactant, (iii) a cationic conditioning polymer, and (iv) a quaternary ammonium group-containing silylated urethane polymer. First, each component will be described.

### (i) Anionic Surfactant

A taurine-derivative surfactant is particularly used for the anionic surfactant to be blended in the composition of the present invention in terms of imparting good use feeling during rinsing (finger-running-through-hair properties and pliability) to the composition and improving the fading-inhibiting effect of the quaternary ammonium group-containing silylated urethane polymer.

Examples of the taurine-derivative surfactant include an N-acyl taurine salt represented by the following formula (I).

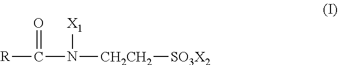

In the formula (I), R represents a linear or branched alkyl group having preferably 10 to 18, more preferably 12 to 14 carbon atoms. X₁ represents a hydrogen atom or a methyl group. Examples of X₂ include a hydrogen atom, an alkali metal, an alkaline earth metal, ammonium, a lower alkanolamine cation, a lower alkylamine cation, and a basic amino acid cation.

Examples of the N-acyl taurine salt include N-lauroyl taurine, N-myristoyl taurine, N-lauroyl methyltaurine sodium salt, N-myristoyl methyltaurine sodium salt, N-stearoyl methyltaurine sodium salt, coconut oil fatty acid methyltaurine sodium salt, palmitoyl methyltaurine sodium salt, and coconut oil fatty acid taurine sodium salt.

Other than the above, examples of the taurine-derivative surfactant include taurine-conjugated bile acid and a salt thereof.

In the present invention, the use of N-lauroyl methyltaurine sodium salt and coconut oil fatty acid methyltaurine sodium salt is suitable among others.

Note that these taurine-derivative surfactants may be used singly or in combinations of two or more.

The amount of the anionic surfactant, that is, taurine-derivative surfactant, blended in the shampoo composition according to the present invention is not particularly limited as long as it is the amount that can exhibit the usual cleaning effect as a shampoo, but in terms of use feeling and fading-inhibiting effect, it is preferably 1 to 20 mass %, more preferably 3 to 12 mass %, relative to the composition. If the amount blended is less than 1 mass %, the use feeling as a shampoo will be insufficient. Further, in the case of the taurine-derivative surfactant, if the amount blended exceeds 20 mass %, not only the use feeling but also the fading-inhibiting effect tends to be reduced because of its high detergency.

### (ii) Amphoteric Surfactant

An alkylamide betaine amphoteric surfactant is particularly used for the amphoteric surfactant to be blended in the composition of the present invention in terms of imparting use feeling during rinsing (finger-running-through-hair properties and pliability) to the composition and improving the fading-inhibiting effect of the quaternary ammonium group-containing silylated urethane polymer.

The alkylamide betaine amphoteric surfactant can be represented, for example, by the following formulas (II) and (III).

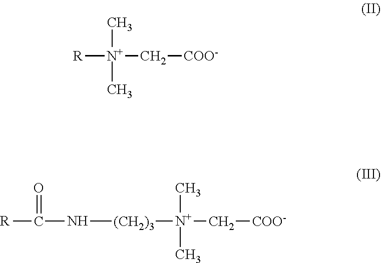

In the formulas (II) and (III), R represents a linear or branched alkyl group having preferably 8 to 18, more preferably 12 to 14 carbon atoms.

Examples of the alkylamide betaine amphoteric surfactants include lauryl dimethylamino acetic acid betaine, palm kernel oil amide propyl dimethylamino acetic acid betaine, and coconut oil fatty acid amide propyl betaine, and these may be used singly or in combinations of two or more. In the present invention, the coconut oil fatty acid amide propyl betaine is particularly preferred.

The amount of the (ii) amphoteric surfactant, that is, alkylamide betaine amphoteric surfactant, blended in the shampoo composition according to the present invention is not particularly limited as long as it is the amount that can exhibit the usual cleaning effect as a shampoo, but in terms of use feeling and fading-inhibiting effect, it is preferably 1 to 20 mass % relative to the composition. If the amount blended is less than 1 mass %, the use feeling as a shampoo will be insufficient, and if the amount blended exceeds 20 mass %, both the use feeling and the fading-inhibiting effect tend to be reduced.

(iii) Cationic Conditioning Polymer

A cationic polymer commonly used for hair cosmetics as a conditioning component can be used as the cationic conditioning polymer to be blended in the composition of the present invention. Examples of such polymers include semisynthetic products from natural polysaccharide such as cationized cellulose, cationized locust bean gum, cationized guar gum, and cationized starch, and synthetic products such as a homopolymer of diallyl quaternary ammonium salt, a diallyl quaternary ammonium salt/acrylamide copolymer, a quaternized polyvinylpyrrolidone derivative, a polyglycol polyamine condensate, a vinyl imidazolium trichloride/vinylpyrrolidone copolymer, a hydroxyethylcellulose/dimethyldiallyl ammonium chloride copolymer, a vinylpyrrolidone/quaternized dimethylaminoethyl methacrylate copolymer, a polyvinylpyrrolidone/alkylamino acrylate copolymer, a polyvinylpyrrolidone/alkylamino acrylate/vinylcaprolactam copolymer, a vinylpyrrolidone/methacrylamide propyltrimethylammonium chloride copolymer, an alkylacrylamide/acrylate/alkylaminoalkylacrylamide/polyethylene glycol methacrylate copolymer, and an adipic acid/dimethylaminohydroxypropyl ethylenetriamine copolymer. Although it is possible to use these cationic conditioning polymers in the present invention, it is particularly preferred to use a cationic polymer having a structure represented by the following formula (IV) in terms of further improving use feeling (finger-running-through-hair properties and pliability) during rinsing.

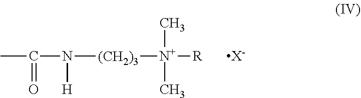

In the formula (IV), R represents an alkyl group having 1 to 3 carbon atoms which may have a group selected from the group consisting of a primary to tertiary amino group, a quaternary ammonium group, and a hydroxyl group; and X⁻ represents a monovalent anion in a number enough to electrically neutralize the structure.

In the above description, the primary amino group is represented by —NH₂; the secondary amino group is represented by —NHR¹; the tertiary amino group is represented by —NHR²R³; and the quaternary ammonium group is represented by —N⁺R⁴R⁵R⁶, where R¹ to R⁶ are each an alkyl group having 1 to 3 carbon atoms, that is, a methyl group, an ethyl group, or a propyl group.

Therefore, examples of the “alkyl group having 1 to 3 carbon atoms which may have a group selected from the group consisting of a primary to tertiary amino group, a quaternary ammonium group, and a hydroxyl group” include a methyl group, an ethyl group, a propyl group, a hydroxymethyl group, a hydroxyethyl group, a hydroxypropyl group, a hydroxypropyl trimethylammonium group (—CH₂CH(OH)CH₂N⁺(CH₃)₃), a hydroxypropyl dimethylamino group (—CH₂CH(OH)CH₂N(CH₃)₂), a hydroxypropyl monomethylamino group (—CH₂CH(OH)CH₂NHCH₃), a hydroxypropylamino group (—CH₂CH(OH)CH₂NH₂), a hydroxypropyl triethylammonium group (—CH₂CH(OH)CH₂N⁺(CH₂CH₃)₃), a hydroxypropyl diethylamino group (—CH₂CH(OH)CH₂N(CH₂CH₃)₂), a hydroxypropyl monoethylamino group (—CH₂CH(OH)CH₂NHCH₂CH₃), a hydroxypropyl tripropylammonium group (—CH₂CH(OH)CH₂N⁺(CH₂CH₂CH₃)₃), a hydroxypropyl dipropylamino group (—CH₂CH(OH)CH₂N(CH₂CH₂CH₃)₂), a hydroxypropyl monopropylamino group (—CH₂CH(OH)CH₂NHCH₂CH₂CH₃), a trimethylpropylammonium group (—CH₂CH₂CH₂N⁺(CH₃)₃), a dimethylpropylamino group (—CH₂CH₂CH₂N(CH₃)₂), a methylpropylamino group (—CH₂CH₂CH₂NHCH₃), a propylamino group (—CH₂CH₂CH₂NH₂), a hydroxyethyl trimethylammonium group (—CH(OH)CH₂N⁺(CH₃)₃), a hydroxyethyl dimethylamino group (—CH(OH)CH₂N(CH₃)₂), a hydroxyethyl monomethylamino group (—CH(OH)CH₂NHCH₃), a hydroxyethylamino group (—CH(OH)CH₂NH₂), a hydroxyethyl triethylammonium group (—CH(OH)CH₂N⁺(CH₂CH₃)₃), a hydroxyethyl diethylamino group (—CH(OH)CH₂N(CH₂CH₃)₂), a hydroxyethyl monoethylamino group (—CH(OH)CH₂NHCH₂CH₃), a hydroxyethyl tripropylamino group (—CH(OH)CH₂N⁺(CH₂CH₂CH₃)₃), a hydroxyethyl dipropylamino group (—CH(OH)CH₂N(CH₂CH₂CH₃)₂), a hydroxyethyl monopropylamino group (—CH(OH)CH₂NHCH₂CH₂CH₃), a trimethylethylammonium group (—CH₂CH₂N⁺(CH₃)₃), a dimethylethylamino group (—CH₂CH₂N(CH₃)₂), a monomethylethylamino group (—CH₂CH₂NHCH₃), an ethylamino group (—CH₂CH₂NH₂), a hydroxymethyl trimethylammonium group (—CH(OH)N⁺(CH₃)₃), a hydroxymethyl dimethylamino group (—CH(OH)N(CH₃)₂), a hydroxymethyl monomethylamino group (—CH(OH)NHCH₃), a hydroxymethylamino group (—CH(OH)NH₂), a hydroxymethyl triethylammonium group (—CH(OH)N⁺(CH₂CH₃)₃), a hydroxymethyl diethylamino group (—CH(OH)N(CH₂CH₃)₂), a hydroxymethyl monoethylamino group (—CH(OH)NHCH₂CH₃), a hydroxymethyl tripropylammonium group (—CH(OH)N⁺(CH₂CH₂CH₃)₃), a hydroxymethyl dipropylamino group (—CH(OH)N(CH₂CH₂CH₃)₂), a hydroxymethyl monopropylamino group (—CH(OH)NHCH₂CH₂CH₃), a trimethyl methylammonium group (—CH₂N⁺(CH₃)₃), a dimethyl methylamino group (—CH₂N(CH₃)₂), a monomethyl methylamino group (—CH₂CH₂NHCH₃), and a methylamino group (—CH₂NH₂).

In the present invention, R is particularly preferably a methyl group or a hydroxypropyl trimethylammonium group.

Examples of the monovalent anion suitable for X⁻ include the ions of halogen atoms such as chlorine, bromine, and iodine, methylsulfuric acid, and ethylsulfuric acid. Note that the number of the anions is set so as to be electrically neutral depending on the number of positive ions in formula (IV).

The structure represented by the formula (IV) can be introduced into a polymer as a side chain of the polymer as constituent monomers such as methacrylamide propyltrimethylammonium chloride (MAPTAC) and acrylamide propyltrimethylammonium chloride (AAPTAC) either by being homopolymerized or by being copolymerized with a common vinyl or acrylic monomer. In the case of the copolymer, the constituent monomer having a structure represented by formula (IV) may be contained in an amount of 1% or more, preferably 10% or more in a molar ratio.

Examples of the cationic polymer having a structure represented by the formula (IV) include a methacrylamide propyltrimethylammonium chloride polymer; an acrylamide/methacrylamide propyltrimethylammonium chloride copolymer; an acrylic acid/methyl acrylate/methacrylamide propyltrimethylammonium chloride copolymer; a trimethylaminopropylacrylamide chloride/dimethylacrylamide copolymer; and Polyquaternium-74 (acrylic acid/methacrylamide propyldimethylammonium chloride/hydroxypropyl trimethylammonium copolymer). One or two or more of these polymers can be suitably used.

Examples of commercially available products of the compound include Merquat 2001 and Merquat 2003 (manufactured by Nalco Japan, Co., Ltd.), DIASLEEK C-822 (manufactured by Mitsubishi Chemical Corporation), and Polyquaternium-74 (manufactured by Rhodia, Inc.).

In the present invention, it is particularly preferable to contain one or two of a trimethylaminopropylacrylamide chloride/dimethylacrylamide copolymer and an acrylic acid/methyl acrylate/methacrylamide propyltrimethylammonium chloride copolymer as the (iii) cationic conditioning polymer.

The amount of the (iii) cationic conditioning polymer blended in the shampoo composition according to the present invention is not particularly limited as long as it is the amount that can exhibit a usual conditioning effect as a shampoo, but it is preferably 0.01 to 2 mass %, more preferably 0.02 to 1 mass %, relative to the composition in terms of further improving the use feeling to the hair after dyeing. If the amount blended is less than 0.01 mass % or exceeds 2 mass %, the finger-running-through-hair properties and pliability during rinsing may be insufficient.

### (IV) Quaternary Ammonium Group-Containing Silylated Urethane Polymer

The quaternary ammonium group-containing silylated urethane polymer may be any urethane polymer having at least one quaternary ammonium group and at least one reactive silyl group. Examples of the reactive silyl group include a hydrolyzable silyl group and a silanol group.

The quaternary ammonium group-containing silylated urethane polymer in the present invention is preferably a compound having a structure including structural units corresponding to the following components (A), (B), and (C), in which a urea bond is formed by combining the following component (D) with an isocyanate terminal of a urethane polymer in which a tertiary amine part derived from the component (C) is converted to a quaternary ammonium ion, among others:

Component (A): a polyisocyanate compound,

Component (B): a polyol compound,

Component (C): a tertiary amine compound having two or more hydroxyl groups, and

Component (D): an ester-modified amino group-containing alkoxysilane represented by the following formula (d1), (d2), or (d3).

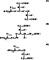

(In the formulas, R¹ and R² may be the same or different and each represent an alkyl group; and R³ and R⁴ may be the same or different and each represent an alkylene group which may have a substituent or an arylene group which may have a substituent. R⁵ represents an alkyl group, a cycloalkyl group, an aryl group, or an aralkyl group; and R⁶ represents a hydrogen atom or —COOR^{6′}, where R^{6′} represents an alkyl group. Further, m is an integer of 1 to 3. When in is 1, two R²s may be the same or different. When m is an integer of 2 or more, two or more R¹O— groups may be the same or different.)

The quaternary ammonium group-containing silylated urethane polymer can be synthesized at least through the following steps (1), (2), and (3):

Step (1): a step of reacting the components (A), (B), and (C) to synthesize a urethane polymer,

Step (2): a step of converting the tertiary amine part derived from the component (C) to a quaternary ammonium ion, and

Step (3): a step of reacting the component (D) with the isocyanate terminal of the urethane polymer.

### [Component (A): Polyisocyanate Compound]

The component (A) may be a compound having at least two isocyanate groups in a molecule, and examples thereof include aliphatic polyisocyanates, alicyclic polyisocyanates, aromatic polyisocyanates, and aromatic-aliphatic polyisocyanates.

Examples of the aliphatic polyisocyanates include aliphatic diisocyanates such as 1,3-trimethylene diisocyanate, 1,4-tetramethylene diisocyanate, 1,3-pentamethylene diisocyanate, 1,5-pentamethylene diisocyanate, 1,6-hexamethylene diisocyanate, 1,2-propylene diisocyanate, 1,2-butylene diisocyanate, 2,3-butylene diisocyanate, 1,3-butylene diisocyanate, 2-methyl-1,5-pentamethylene diisocyanate, 3-methyl-1,5-pentamethylene diisocyanate, 2,4,4-trimethyl-1,6-hexamethylene diisocyanate, 2,2,4-trimethyl-1,6-hexamethylene diisocyanate, 2,6-diisocyanate methylcaproate, and lysine diisocyanate.

Examples of the alicyclic polyisocyanates include alicyclic diisocyanates such as 1,3-cyclopentane diisocyanate, 1,4-cyclohexane diisocyanate, 1,3-cyclohexane diisocyanate, 4,4′-methylenebis(cyclohexyl isocyanate), methyl-2,4-cyclohexane diisocyanate, methyl-2,6-cyclohexane diisocyanate, 1,3-bis(isocyanatemethyl)cyclohexane, 1,4-bis(isocyanatemethyl)cyclohexane, isophorone diisocyanate (IPDI), and norbornane diisocyanate.

Examples of the aromatic polyisocyanates include aromatic diisocyanates such as m-phenylene diisocyanate, p-phenylene diisocyanate, 2,4-tolylene diisocyanate, 2,6-tolylene diisocyanate, naphthylene-1,4-diisocyanate, naphthylene-1,5-diisocyanate, 4,4′-diphenyl diisocyanate, 4,4′-diphenylether diisocyanate, 2-nitrodipheny-4,4′-diisocyanate, 2,2′-diphenylpropane-4,4′-diisocyanate, 3,3′-dimethyldiphenylmethane-4,4′-diisocyanate, 4,4′-diphenylpropane diisocyanate, and 3,3′-dimethoxydiphenyl-4,4′-diisocyanate.

Examples of the aromatic aliphatic polyisocyanates include aromatic aliphatic diisocyanates such as 1,3-xylylene diisocyanate, 1,4-xylylene diisocyanate, ω,ω′-diisocyanate-1,4-diethylbenzene, 1,3-bis(1-isocyanate-1-methylethyl)benzene, 1,4-bis(1-isocyanate-1-methylethyl)benzene, and 1,3-bis(α,α-dimethylisocyanatemethyl)benzene.

Further examples include dimers and trimers, reaction products, and polymers of the aliphatic polyisocyanates, alicyclic polyisocyanates, aromatic polyisocyanates, and aromatic aliphatic polyisocyanates (such as a dimer and a trimer of diphenylmethane diisocyanate, a reaction product of trimethylolpropane with tolylene diisocyanate, a reaction product of trimethylolpropane with hexamethylene diisocyanate, polymethylene polyphenol isocyanate, polyether polyisocyanate, and polyester polyisocyanate).

Examples of the component (A) which can be suitably used in the present invention include 1,6-hexamethylene diisocyanate, 4,4′-methylenebis (cyclohexyl isocyanate), 1,3-bis(isocyanatemethyl)cyclohexane, 1,4-bis(isocyanatemethyl)cyclohexane, isophorone diisocyanate (IPDI), 2,4-tolylene diisocyanate, 2,6-tolylene diisocyanate, 4,4′-diphenylmethane diisocyanate, 1,3-xylylene diisocyanate, 1,4-xylylene diisocyanate, norbornane diisocyanate, and 1,3-bis(α,α-dimethylisocyanatemethyl)benzene, among others. These may be used singly or in combinations of two or more. Note that when an aliphatic polyisocyanate is used, a resin with little discoloration can be obtained.

### [Component (B): Polyol Compound]

The component (B) may be a compound having two or more hydroxyl groups, and examples thereof include polyhydric alcohols, polyether polyols, polyester polyols, polycarbonate polyols, polyolefin polyols, polyacrylic polyols, polyalkylene oxide adducts of tri- or higher polyhydric alcohols or derivatives thereof in which terminal hydroxyl groups are sealed, and castor oil. Compounds selected from polyether polyols, polyester polyols, polycarbonate polyols, and polyalkylene oxide adducts of tri- or higher polyhydric alcohols or derivatives thereof in which terminal hydroxyl groups are sealed are preferred, among others, as the component (B) in the present invention in terms of relatively easy handling during production.

Examples of the polyether polyols include polyalkylene glycols such as polyethylene glycol, polypropylene glycol, and polytetramethylene ether glycol (PTMG); and (alkylene oxide-another alkylene oxide) copolymers containing a plurality of alkylene oxides such as an ethylene oxide-propylene oxide copolymer. In the present invention, commercially available products such as trade name “PTMG 2000” (manufactured by Mitsubishi Chemical Corporation) may be used.

Examples of the polyester polyols which can be used include condensation polymers of a polyhydric alcohol and a polyvalent carboxylic acid; ring-opened polymers of a cyclic ester (lactone); and reaction products of three components of a polyhydric alcohol, a polyvalent carboxylic acid, and a cyclic ester. These can be used singly or in combinations of two or more.

Examples of the polyhydric alcohols include ethylene glycol, diethylene glycol, propylene glycol, dipropylene glycol, trimethylene glycol, 1,4-tetramethylenediol, 1,3-tetramethylenediol, 2-methyl-1,3-trimethylenediol, 1,5-pentamethylenediol, neopentyl glycol, 1,6-hexamethylenediol, 3-methyl-1,5-pentamethylenediol, 2,4-diethyl-1,5-pentamethylenediol, glycerin, trimethylolpropane, trimethylolethane, cyclohexanediols (such as 1,4-cyclohexanediol), bisphenols (such as bisphenol A), and sugar alcohols (such as xylitol and sorbitol). Examples of the polyvalent carboxylic acids include aliphatic dicarboxylic acids such as malonic acid, maleic acid, succinic acid, glutaric acid, adipic acid, suberic acid, azelaic acid, sebacic acid, and dodecanedioic acid; alicyclic dicarboxylic acids such as 1,4-cyclohexanedicarboxylic acid; and aromatic dicarboxylic acids such as terephthalic acid, isophthalic acid, orthophthalic acid, 2,6-naphthalene dicarboxylic acid, paraphenylene dicarboxylic acid, and trimellitic acid. Further, examples of the cyclic esters include propiolactone, β-methyl-δ-valerolactone, and ε-caprolactone.

Examples of the polycarbonate polyols include reaction products of a polyhydric alcohol with phosgene; and ring-opened polymers of a cyclic carbonate. These can be used singly or in combinations of two or more. Examples of the polyhydric alcohols used for the reaction of a polyhydric alcohol with phosgene include the same examples as in the polyhydric alcohols as described above. Examples of the cyclic carbonates include alkylene carbonates such as ethylene carbonate, trimethylene carbonate, tetramethylene carbonate, and hexamethylene carbonate. Note that polycarbonate polyols in the present invention may be a compound having a carbonate bond in the molecule with a terminal hydroxyl group, and the compound may have an ester bond together with the carbonate bond.

The polyalkylene oxide adducts of tri- or higher polyhydric alcohols or derivatives thereof in which terminal hydroxyl groups are sealed include compounds obtained by adding a polyalkylene oxide to one of the hydroxyl groups which a tri- or higher polyhydric alcohol has and derivatives in which terminal hydroxyl groups of the adducts are sealed with an alkyl group such as a methyl or ethyl group or an acyl group such as acetyl or benzoyl group.

Examples of the tri- or higher polyhydric alcohols include trimethylolpropane, trimethylolethane, glycerin, pentaerythritol, xylitol, and sorbitol. These may be used singly or in combinations of two or more. In the present invention, trimethylolpropane and trimethylolethane are preferred among others.

Further, the polyalkylene oxides include alkylene oxide derivatives containing a single alkylene oxide and (alkylene oxide-another alkylene oxide) copolymers containing a plurality of alkylene oxides. Examples of the alkylene oxide include aliphatic epoxides such as alkylene oxides having 2 to 8 carbon atoms such as ethylene oxide, propylene oxide, butylene oxide, isobutylene oxide, 1-butene oxide, 2-butene oxide, trimethylethylene oxide, tetramethylene oxide, tetramethylethylene oxide, butadiene monoxide, and octylene oxide, and, in addition, dipentaneethylene oxide and dihexaneethylene oxide; alicyclic epoxides such as trimethylene oxide, tetramethylene oxide, tetrahydropyran, tetrahydropyran, and octylene oxide; and aromatic epoxides such as styrene oxide and 1,1-diphenylethylene oxide. The polyalkylene oxide of the present invention preferably contains, among others, an alkylene oxide having 2 to 4 carbon atoms such as ethylene oxide and propylene oxide, particularly ethylene oxide, in terms of providing excellent water-dispersion stability.

Examples of the polyalkylene oxide adducts of tri- or higher polyhydric alcohols or derivatives thereof in which terminal hydroxyl groups are sealed in the present invention include trimethylolpropane mono(polyalkylene oxide alkyl ether) such as trimethylolpropane mono(polyethylene oxide methyl ether) and trimethylolpropane mono(polyethylene oxide ethyl ether); polyoxyalkylene sorbitan mono-fatty acid ester such as polyoxyethylene sorbitan monolaurate and polyoxyethylene sorbitan monostearate; polyoxyethylene glyceryl mono-fatty acid ester such as polyoxyethylene glyceryl monolaurate and polyoxyethylene glyceryl monostearate; and trimethylolpropane mono(polyalkylene oxide alkyl ether) such as trimethylolpropane mono(polyethylene oxide methyl ether).

In the present invention, trimethylolpropane mono(polyalkylene oxide alkyl ether) is preferred among others, and a compound represented by the following formula (b) is particularly preferred.

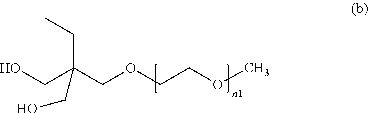

(In the formula, n1 represents an integer of 10 to 40.) In the present invention, commercially available products such as trade name “Ymer N120” (manufactured by Perstorp Inc.) may be used.

The number average molecular weight of the component (B) in the present invention is preferably about 500 to 5000, more preferably about 800 to 3000. If the number average molecular weight is less than 500, the fading-inhibiting effect tends to be reduced. On the other hand, if the number average molecular weight exceeds 5000, the water-dispersion stability tends to be reduced.

The component (B) in the present invention preferably includes one or more compounds selected from polyether polyols, polyester polyols, polycarbonate polyols, and polyalkylene oxide adducts of tri- or higher polyhydric alcohols or derivatives thereof in which terminal hydroxyl groups are sealed, and particularly preferably includes at least polyalkylene oxide adducts of tri- or higher polyhydric alcohols or derivatives thereof in which terminal hydroxyl groups are sealed (in particular, a compound represented by the formula (b)) in that the adsorptivity to the hair surface is further improved, and the fading-inhibiting effect can be further improved by introducing pendant nonionic side chains (hydrophilic groups) into urethane polymers.

The proportion of the polyalkylene oxide adducts of tri- or higher polyhydric alcohols or derivatives thereof in which terminal hydroxyl groups are sealed in the component (B) is for example 5 to 100 mass %, preferably 10 to 50 mass %, and particularly preferably 20 to 40 mass %.

### [Component (C): Tertiary Amine Compound Having Two or More Hydroxyl Groups]

The component (C) may be a compound having a cationizable tertiary amine and two or more hydroxyl groups, and examples thereof include trialkanol amines such as triethanolamine, tri-n-propanolamine, and tri-iso-propanolamine; and N-hydrocarbon group-substituted-dialkanolamines such as N-methyl diethanolamine and N-phenyl diethanolamine.

As the component (C) in the present invention, N-hydrocarbon group-substituted-N,N-dialkanolamine is preferred among others, and
N-methyl-N,N-diethanolamine, N-ethyl-N,N-diethanolamine, N-methyl-N,N-dipropanolamine are particularly preferred.

### [Component (D): Ester-Modified Amino Group-Containing Alkoxysilane]

The component (D) in the present invention is represented by the formula (d1), (d2), or (d3). In the formulas, R¹ and R² may be the same or different and each represent an alkyl group; and R³ and R⁴ may be the same or different and each represent an alkylene group which may have a substituent or an arylene group which may have a substituent. R⁵ represents an alkyl group, a cycloalkyl group, an aryl group, or an aralkyl group; and R⁶ represents a hydrogen atom or —COOR^{6′}, where R^{6′} represents an alkyl group. Further, m is an integer of 1 to 3. When m is 1, two R²s may be the same or different. When m is an integer of 2 or more, two or more R¹O— groups may be the same or different.

R¹ and R² in the formulas (d1), (d2), and (d3) may be the same or different and each represent an alkyl group. Examples of the alkyl group include a methyl, ethyl, propyl, isopropyl, butyl, isobutyl, s-butyl, t-butyl, pentyl, isopentyl, s-pentyl, t-pentyl, hexyl, isohexyl, s-hexyl, t-hexyl, heptyl, octyl, nonyl, decyl, undecyl, dodecyl, tridecyl, tetradecyl, pentadecyl, hexadecyl, heptadecyl, octadecyl, nonadecyl, and eicosyl group. As the alkyl group for R¹ and R² of the present invention, an alkyl group having one to about 6 carbon atoms is preferred (particularly an ethyl group in terms of safety) among others.

R³ and R⁴ in the formulas (d1), (d2), and (d3) may be the same or different and each represent an alkylene group which may have a substituent or an arylene group which may have a substituent. Examples of the alkylene group include a methylene, ethylene, trimethylene, tetramethylene, pentamethylene, decamethylene, and tetradecamethylene group. As the alkylene group for R³ and R⁴ of the present invention, an alkylene group having 1 to 10 carbon atoms is preferred among others. Examples of the arylene group include a phenylene, naphthylene, and anthrylene group. As the arylene group for R³ and R⁴ of the present invention, an alkylene group having 6 to 10 carbon atoms is preferred among others.

Further, examples of the substituent which R³ and R⁴ may have include aryl groups such as a phenyl group; alkyl groups such as a methyl group, an ethyl group, a propyl group, and a butyl group; and cycloalkyl groups such as a cyclohexyl group. Further, the substituent may further have other substituents (such as an alkoxy group, an aryloxy group, a cycloalkyloxy group, an alkoxycarbonyl group, an aryloxycarbonyl group, a cycloalkyloxycarbonyl group, an acyl group, and an amino group).

R⁵ in the formulas (d1), (d2), and (d3) represents an alkyl group, a cycloalkyl group, an aryl group, or an aralkyl group. Examples of the alkyl group include the same examples as in the alkyl group for R¹ and R². As the alkyl group for R⁵ of the present invention, an alkyl group having 1 to 20 carbon atoms is preferred among others. Examples of the cycloalkyl group include monocyclic, polycyclic, or condensed cycloalkyl groups having 3 to 20 carbon atoms such as a cyclopropyl, cyclopentyl, cyclohexyl, and cyclooctyl group. Examples of the aryl group include aryl groups having 6 to 20 carbon atoms such as a phenyl, tolyl, xylyl, naphthyl, methylnaphthyl, anthryl, phenanthryl, and biphenyl group. Examples of the aralkyl group include the alkyl group substituted with the aryl group.

R⁶ in the formulas (d1), (d2), and (d3) represents a hydrogen atom or —COOR^{6′}, where R^{6′} represents an alkyl group. Examples of the alkyl group for R^{6′} include the same examples as in the alkyl group for R¹ and R², and an alkyl group having 1 to 20 carbon atoms is preferred among others.

The ester-modified amino group-containing alkoxysilane represented by the formulas (d1), (d2), and (d3) can be synthesized, for example, by the Michael addition reaction of a nitrogen atom of the primary or secondary amino group in a primary or secondary amino group-containing alkoxysilane compound represented by the following formula (d1, 2-1):

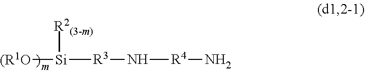

(In the formula, R¹, R², R³, R⁴, and m are the same as in the formulas (d1) to (d3)) or in a primary amino group-containing alkoxysilane compound represented by the following formula (d3-1):

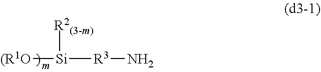

(In the formula, R¹, R², R³, and m are the same as in the formulas (d1) to (d3).) with the unsaturated bond (carbon-carbon double bond) of an unsaturated carboxylate represented by the following formula (1):

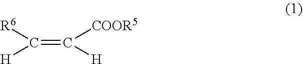

(In the formula, R⁵ and R⁶ are the same as in the formulas (d1) to (d3)). The Michael addition reaction can be carried out in the presence or absence of a solvent. Further, application of heat or pressure may be performed during the reaction.

Examples of the primary and secondary amino group-containing alkoxysilane compounds represented by formula (d1, 2-1) include N-(aminoalkyl)aminoalkyltrialkoxysilane such as N-β(aminoethyl)-γ-aminopropyltrimethoxysilane and N-β(aminoethyl)-γ-aminopropyltriethoxysilane; and N-(aminoalkyl)aminoalkylalkyldialkoxysilane such as N-β(aminoethyl)-γ-aminopropylmethyldimethoxysilane and N-β(aminoethyl)-γ-aminopropylmethyldiethoxysilane. In the present invention, commercially available products such as trade names “KBE602”, “KBM602”, “KBE603”, and “KBM603” (all manufactured by Shin-Etsu Chemical Co., Ltd.) may be used.

Examples of the primary amino group-containing alkoxysilane compound represented by formula (d3-1) include aminoalkyltrialkoxysilanes such as aminomethyltrimethoxysilane, aminomethyltriethoxysilane, 2-aminoethyltrimethoxysilane, 2-aminoethyltriethoxysilane, 3-aminopropyltrimethoxysilane, 3-aminopropyltriethoxysilane, 3-aminopropyltripropoxysilane, 3-aminopropyltriisopropoxysilane, and 3-aminopropyltributoxysilane; and (aminoalkyl)alkoxysilanes such as 2-aminoethylmethyldimethoxysilane, 2-aminoethylmethyldiethoxysilane, and 3-aminopropylmethyldipropoxysilane. In the present invention, commercially available products such as trade names “KBE902”, “KBM902”, “KBE903”, and “KBM903” (all manufactured by Shin-Etsu Chemical Co., Ltd.), may be used.

Examples of the unsaturated carboxylate represented by the formula (1) include n-butyl acrylate, isobutyl acrylate, cyclohexyl acrylate, 2-ethylhexyl acrylate, 3-butyl cyclohexyl acrylate, lauryl acrylate, cetyl acrylate, stearyl acrylate, behenyl acrylate, and glycidyl acrylate. As the unsaturated carboxylate represented by formula (1) in the present invention, n-butyl acrylate, lauryl acrylate, 2-ethylhexyl acrylate, and the like are preferred among others.

### [Step (1): Synthesis of Urethane Polymer]

Urethane polymers can be synthesized by reacting the components (A), (B), and (C) according to known or conventional methods for preparing urethane polymers from a polyol compound and a polyisocyanate compound. A polymerization catalyst may be used for the synthesis of urethane polymers for accelerating the reaction.

A known or conventional polymerization catalyst (curing catalyst) used for the reaction of a polyol compound with a polyisocyanate compound can be used as the above polymerization catalyst, and examples thereof include a basic compound such as an amine compound. Examples of the basic compound such as an amine compound include aminosilanes such as γ-aminopropyl trimethoxysilane and γ-aminopropyl triethoxysilane; quaternary ammonium salts such as tetramethyl ammonium chloride and benzalkonium chloride; and linear or cyclic tertiary amines or quaternary ammonium salts containing a plurality of nitrogen atoms such as trade names “DABCO” series and “DABCO BL” series manufactured by Sankyo Air Products Co., Ltd, and 1,8-diazabicyclo[5.4.0]undec-7-ene (DBU).

The reaction can be carried out in a solvent. Examples of the solvent include acetone, methyl ethyl ketone, methyl isobutyl ketone, N-methylpyrrolidone, tetrahydrofuran, and ethyl acetate. The atmosphere during the reaction is not particularly limited, but is selected from an air atmosphere, a nitrogen atmosphere, an argon atmosphere, and the like. The reaction temperature can be suitably selected depending on the type of reaction components and the like and is for example about 20 to 150° C., preferably about 20 to 100° C. The reaction may be carried out under normal pressure or may be carried out under reduced pressure or pressurization. The reaction time can be suitably selected depending on the reactivity of the components and is for example about 2 to 20 hours, preferably about 3 to 10 hours.

The amount of the components (A), (B), and (C) to be used is not particularly limited and can be suitably adjusted depending on the various physical properties to be determined, and the isocyanate group in the component (A)/the hydroxyl group in the components (B) and (C) (NCO group/OH group) (equivalent ratio) is, for example, in a range of greater than 1 and 1.5 or less (preferably greater than 1 and 1.3 or less, more preferably greater than 1 and 1.2 or less). If the ratio of the NCO group/OH group is too large (for example, if it exceeds 1.5 (equivalent ratio)), the dispersibility tends to be reduced. On the other hand, if the ratio of the NCO group/OH group is too small (for example, if it is 1 or less (equivalent ratio)), the introduction of silyl groups cannot sufficiently be carried out, and the fading-inhibiting effect tends to be reduced.

Further, it is preferred that the component (C) be contained in a proportion such that the content of a cationizable tertiary amine in the urethane polymer is 2 to 90 mass % (preferably 2 to 50 mass %, more preferably 5 to 20 mass %). If the content of the cationizable tertiary amine exceeds the above range, the viscosity tends to be too high to make its use easy. On the other hand, if the content of the cationizable tertiary amine is less than the above range, the water-dispersion stability tends to be reduced.

It is preferred that the content of a terminal isocyanate group of the urethane polymer be about 0.3 to 7.0 weight %, for example. If the content of the terminal isocyanate group exceeds 7 weight %, the water dispersion tends to be difficult to achieve. On the other hand, if the content of the terminal isocyanate group is less than 0.3 weight %, the viscosity during the synthesis tends to be too high to make the synthesis easy.

### [Step (2): Conversion of Tertiary Amine Part to Quaternary Ammonium Ion]

A quaternary ammonium group-containing urethane polymer can be synthesized by converting the tertiary amine part derived from the component (C) in the urethane polymer obtained through the step (1) to a quaternary ammonium ion (cationization).

Examples of the method for cationizing the nitrogen atom of a tertiary amine include a method of reacting an alkylating agent (quaternizing agent) with the urethane polymer obtained through the step (1) to introduce, into the tertiary amine part derived from the component (C), an alkyl group having 1 to 20 carbon atoms such as methyl, ethyl, propyl, butyl, and pentyl; an alkenyl group having 2 to 20 carbon atoms such as a vinyl, isopropenyl, allyl, metallyl, 3-butenyl, 2-methyl-1-butenyl, 3-methyl-1-butenyl, 2-methyl-3-butenyl, and 3-methyl-3-butenyl group; an aralkyl group having 7 to 11 carbon atoms such as benzyl and 2-phenylethyl group; and the like.

Examples of the alkylating agent (quaternizing agent) include sulfates such as dimethyl sulfate and diethyl sulfate; and halides such as methyl chloride, methyl bromide, methyl iodide, benzyl chloride, and benzyl bromide.

The amount of the alkylating agent to be used can be suitably adjusted and is in a range of, for example, 30 mol % or more (preferably 50 to 120 mol %, more preferably 80 to 100 mol %) relative to 1 mol of the tertiary amine part (tertiary amino group) in the urethane polymer. If the amount of the alkylating agent to be used exceeds the above range, the heat increase during reaction tends to be intense to reduce workability. On the other hand, if the amount of the alkylating agent to be used is less than the above range, the fading-inhibiting effect tends to be reduced.

Further, the cationization reaction can be carried out in a solvent. Examples of the solvent include acetone, methyl ethyl ketone, methyl isobutyl ketone, N-methyl pyrrolidone, tetrahydrofuran, and ethyl acetate. The atmosphere during the reaction is not particularly limited, but is selected from an air atmosphere, a nitrogen atmosphere, an argon atmosphere, and the like. The reaction temperature can be suitably selected depending on the type of reaction components and the like and is for example about 0 to 100° C., preferably about 20 to 80° C. The reaction may be carried out under normal pressure or may be carried out under reduced pressure or pressurization. The reaction time can be suitably selected depending on the reaction rate and is for example about 10 minutes to 5 hours, preferably about 30 minutes to 3 hours.

### [Step (3): Silylation]

A quaternary ammonium group-containing silylated urethane polymer can be synthesized by adding the component (D) to the isocyanate terminal of the quaternary ammonium group-containing urethane polymer obtained in the step (2) (silylation reaction). Note that the treatment of the step (3) may be carried out before applying the treatment of the step (2). In this case, a quaternary ammonium group-containing silylated urethane polymer can be synthesized by synthesizing a silylated urethane polymer by adding the component (D) to the isocyanate terminal of the urethane polymer obtained in the step (1), followed by converting the nitrogen atom of the tertiary amine part in the step (2) to a quaternary ammonium ion. Hereinafter, a method of carrying out the step (2) followed by the step (3) will be described, but the method can be applied to the case of carrying out the step (3) followed by the step (2).

The silylation reaction can be carried out by mixing the quaternary ammonium group-containing urethane polymer obtained in the step (2) and the component (D) and optionally heating the mixture. By carrying out the silylation reaction, the isocyanate group at the terminal of the quaternary ammonium group-containing urethane polymer is combined with the ester-modified alkoxysilane to obtain a quaternary ammonium group-containing silylated urethane polymer. In the silylation reaction, a polymerization catalyst may be optionally used. Further, this reaction can be carried out in the presence or absence of a solvent.

It is preferred that the component (D) be added in a proportion such that the content of the silicon atom in the quaternary ammonium group-containing silylated urethane polymer is 0.05 to 10 mass % (preferably 0.05 to 5 mass %, more preferably 0.05 to 2 mass %). If the silicon content exceeds the above range, the storage stability tends to be reduced, and on the other hand, if the silicon content is less than the above range, the fading-inhibiting effect tends to be reduced.

The atmosphere during the silylation reaction is not particularly limited, but is selected from an air atmosphere, a nitrogen atmosphere, an argon atmosphere, and the like. The reaction temperature can be suitably selected depending on the type of reaction components and the like and is for example about 20 to 100° C., preferably about 40 to 80° C. The reaction may be carried out under normal pressure or may be carried out under reduced pressure or pressurization. The reaction time can be suitably selected and is for example about 10 minutes to 3 hours, preferably about 20 minutes to 2 hours.

### [Step (4): Silicone Chain Addition]

There may be provided a step (step (4)) of further reacting a compound having a hydrolyzable silicon atom-containing group with the terminal alkoxysilyl group derived from the component (D) of the quaternary ammonium group-containing silylated urethane polymer obtained through the steps (1) to (3) to add a silicone chain to the quaternary ammonium group-containing silylated urethane polymer. By adding the silicone chain to the quaternary ammonium group-containing silylated urethane polymer, the adsorptivity to the hair surface can be improved, and the fading-inhibiting effect can be further improved.

The compound having a hydrolyzable silicon atom-containing group is not particularly limited as long as it is a compound having at least one hydrolyzable silicon atom-containing group in the molecule. Examples of the hydrolyzable silicon atom-containing group include hydrolyzable silyl groups such as alkoxysilyl groups, hydrosilyl groups, and halogenated silyl groups (such as a chlorosilyl group, a bromosilyl group, a iodosilyl group, and a fluorosilyl group). Note that one to three (preferably two or three) groups or atoms (such as alkoxy groups, hydrogen atoms, and halogen atoms) are generally bonded to one silicon atom in the hydrolyzable silyl group, where the same groups (particularly alkoxy groups) and atoms may be bonded, or two or more different groups and atoms may be bonded in combination.

As the hydrolyzable silyl group in the present invention, an alkoxysilyl group and a hydrosilyl group are preferred, and an alkoxysilyl group is particularly preferred. As a compound having at least one alkoxysilyl group in the molecule, a compound (E) represented by the following formula (e1) or (e2):

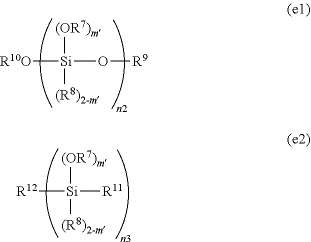

can be suitably used.

In formula (e1), R⁷, R⁸, R⁹, and R¹⁹ may be the same or different and each represent a hydrogen atom or an alkyl group. m′ is 1 or 2. n2 is an integer of 1 or more. Further, in formula (e2), R¹¹ represents (OR⁷) or R⁸, and R¹² represents an organic group. n3 is an integer of 1 or more. R⁷, R⁸, and m′ are the same as the above.

Examples of the alkyl groups for R⁷, R⁸, R⁹, and R¹⁰ include the same examples of the alkyl groups for R¹ and R², and preferred is an alkyl group having 1 to 10 (more preferably 1 to 6, particularly preferably 1 to 4) carbon atoms among others.

Further, the alkyl groups of R⁷ and R⁸ may have a substituent. Further, the alkyl groups of R⁷ and R⁸ may be bonded to other alkyl groups (such as alkyl groups of R⁷ and R⁸ bonded to other silicon atoms) through the substituent to form a ring (an aromatic ring or a non-aromatic ring). Furthermore, R⁷ and R⁸ may be bonded to R⁷ and R⁸ which are bonded to the same or different silicon atom, respectively.

m′ is 1 or 2 and is preferably 2. Note that when m′ is 2, R⁸ is not present, which means that two (OR⁷) groups are bonded to the silicon atom in formula (e1). n2 is an integer of 1 or more. The compound represented by the formula (e1) means a monomer when n2 is 1, and it means a multimer such as oligomer or polymer when n2 is an integer greater than or equal to 2.

Examples of the compound represented by the formula (e1) include monomer compounds such as tetraalkoxysilanes such as tetramethoxysilane, tetraethoxysilane, tetrapropoxysilane, tetraisopropoxysilane, and tetrabutoxysilane; alkoxytrialkoxysilanes such as methoxytriethoxysilane; and dialkoxydialkoxysilanes such as dimethoxydiethoxysilane; and multimer compounds such as polytetraalkoxysilanes such as polytetramethoxysilane, polytetraethoxysilane, polytetrapropoxysilane, polytetraisopropoxysilane, and polytetrabutoxysilane; poly(alkoxyalkoxysilane)s such as poly(methoxyethoxysilane); poly(alkoxysilanes) such as poly(methoxysilane) and poly(butoxysilane); and poly(alkoxyalkylsilanes) such as poly(methoxymethylsilane), poly(methoxyethylsilane), and poly(ethoxymethylsilane).

In the formula (e2), R¹¹ is OR⁷ or R⁸, and R⁷, R⁸, and m′ are the same as R⁷, R⁸, and m′ in formula (e1). Further, a plurality of OR⁷ and R⁸ bonded to the same silicon atom may be the same or different from each other.

Further, examples of the organic group of R¹² include an alkyl group which may have a substituent and a hetero atom-containing group having an atom other than a carbon atom (such as an oxygen atom, a nitrogen atom, and a sulfur atom) in the main chain of the alkyl group, and the alkyl groups which may have a substituent and the hetero atom-containing group may have any of a monovalent or polyvalent form. Examples of the organic group of R¹² include a vinyl group and a mercapto group, and, in addition, a vinyl-alkyl group, a vinyl-(alkyl)-aryl group, a vinyl-(alkyl)-cycloalkyl group, a (meth)acryloyl group, a (meth)acryloyloxyalkyl group (a vinyl-carbonyloxyalkyl group), a (meth)acryloyloxyaryl group, a mercapto-alkyl group, a mercapto-(alkyl)-aryl group, and a mercapto-(alkyl)-cycloalkyl group.

n3 is an integer of 1 or more, and is preferably an integer of 1 to 4 (more preferably 1 or 2, particularly preferably 1). When n3 is an integer of 2 or more, it means that two or more hydrolyzable silicon atom-containing groups are bonded to the organic group of R¹².

Examples of the compounds in which R¹² is an alkyl group among the compounds represented by the formula (e2) include alkyltrialkoxysilanes such as methyltrimethoxysilane, ethyltrimethoxysilane, and methyltriethoxysilane, dialkyldialkoxysilanes such as dimethyldimethoxysilane, dimethyldiethoxysilane, diethyldiethoxysilane, diisopropyldimethoxysilane, isopropyldimethoxymethylsilane, and isopropyldiethoxymethylsilane, and trialkylalkoxysilanes corresponding to these.

Further, examples of the compounds in which R¹² is an alkyl group having a substituent (such as a glycidoxy group, an isocyanate group, and an amino group) include the compounds corresponding to those as illustrated as the compounds in which R¹² is an alkyl group.

Examples of the compounds in which R¹² is a vinyl group among the compounds represented by the formula (e2) include vinyltrialkoxysilanes such as vinyltrimetoxysilane and vinyltriethoxysilane; (vinyl)alkyldialkoxysilanes such as vinylmethyldimethoxysilane and vinylmethyldiethoxysilane, and (vinyl)dialkyl(mono)alkoxysilanes corresponding to these.

Examples of the compounds in which R¹² is a (meth)acryloyloxyalkyl group among the compounds represented by the formula (e2) include (meth)acryloxyalkyl-trialkoxysilanes such as 3-(meth)acryloxypropyl-trimethoxysilane and 3-(meth)acryloxypropyl-triethoxysilane; (meth)acryloxyalkyl-alkyldialkoxy silanes such as 3-(meth)acryloxypropyl-methyldimethoxysilane and 3-(meth)acryloxypropyl-methyldiethoxysilane, and (meth)acryloxyalkyl-dialkyl(mono)alkoxysilanes corresponding to these.

Examples of the compounds in which R¹² is a mercapto-alkyl group among the compounds represented by the formula (e2) include mercaptoalkyl trialkoxysilanes such as 3-mercaptopropyl trimethoxysilane and 3-mercaptopropyl triethoxysilane; (mercaptoalkyl)alkyldialkoxysilanes such as 3-mercaptopropyl methyldipropoxysilane and 3-mercaptopropyl methyldiisopropoxysilane, and (mercaptoalkyl)dialkyl(mono)alkoxysilanes corresponding to these.

Examples of the compounds having a dialkoxysilyl group which can be suitably used include dialkyldialkoxysilanes such as dimethyldimethoxysilane, dimethyldiethoxysilane, dimethyldipropoxysilane, diethyldimethoxysilane, diethyldiethoxysilane, diethyldipropoxysilane, isopropyldimethoxymethylsilane, and isopropyldiethoxymethylsilane; (vinyl)alkyldialkoxysilanes such as vinylmethyldimethoxysilane and vinylmethyldiethoxysilane; and (meth)acryloxyalkyl-alkyldialkoxysilanes such as 3-methacryloxypropylmethyldimethoxysilane and 3-methacryloxypropylmethyldiethoxysilane, among others.

Examples of the compounds having a trialkoxysilyl group which can be suitably used include alkyltrialkoxysilanes such as methyltrimethoxysilane, methyltriethoxysilane, methyltripropoxysilane, ethyltrimethoxysilane, ethyltriethoxysilane, ethyltripropoxysilane, isopropyltrimethoxysilane, and isopropyltriethoxysilane; vinyltrialkoxysilanes such as vinyltrimetoxysilane and vinyltriethoxysilane; (meth)acryloxyalkyl-trialkoxysilanes such as 3-(meth)acryloxypropyl-trimethoxysilane and 3-(meth)acryloxypropyl-triethoxysilane.

It is preferred that the amount of the compound (E) to be used be in a proportion such that the compound (E) is, for example, 1 to 50 mol (preferably 5 to 40 mol, more preferably 5 to 20 mol) relative to 1 mol of silyl groups in a quaternary ammonium group-containing silylated urethane polymer. If the amount of the compound (E) to be used exceeds the above range, the storage stability tends to be reduced.

The atmosphere during the silicone chain addition reaction is not particularly limited, but is selected from an air atmosphere, a nitrogen atmosphere, an argon atmosphere, and the like. The reaction temperature can be suitably selected depending on the type of reaction components and the like and is for example about 20 to 100° C., preferably about 40 to 80° C. The reaction may be carried out under normal pressure or may be carried out under reduced pressure or pressurization. The reaction time can be suitably selected and is for example about 1 to 20 hours, preferably about 1 to 5 hours.

The amount of the (iv) quaternary ammonium group-containing silylated urethane polymer blended in the shampoo composition according to the present invention is not particularly limited, but when the fading-inhibiting effect is taken into consideration, it is preferably 0.01 to 1 mass %, more preferably 0.05 to 0.6 mass % relative to the composition. If the amount blended is less than 0.01 mass %, the fading-inhibiting effect will be insufficient, and if it exceeds 1 mass %, the use feeling may be reduced, for example, stiff hair after application.

In addition to the components (i) to (iv), the shampoo composition according to the present invention can be blended with other components generally used for cosmetics or drugs in the range which does not impair the effect of the present invention, and then can be produced by a conventional method.

Examples of other components include oil, a cationic surfactant, a nonionic surfactant, a powder constituent, a moisturizer, a natural polymer, a synthetic polymer, an ultraviolet absorber, a sequestering agent, a pH adjuster, a skin nutrient, vitamin, an antioxidant, an auxiliary antioxidant, perfume, and water.

Examples of oils include liquid oils and fats, solid oils and fats, hydrocarbon oil, and silicone oil.

Examples of liquid oils and fats include avocado oil, camellia oil, turtle oil, macadamia nut oil, corn oil, mink oil, olive oil, rapeseed oil, egg-yolk oil, sesame oil, persic oil, wheat germ oil, sasanqua oil, castor oil, linseed oil, safflower oil, cottonseed oil, perilla oil, soybean oil, peanut oil, tea oil, Japanese nutmeg oil, rice bran oil, China wood oil, tung oil, jojoba oil, germ oil, and triglycerin.

Examples of solid oils and fats include cacao butter, coconut oil, horse fat, hydrogenated coconut oil, palm oil, beef tallow, mutton tallow, hydrogenated beef tallow, palm kernel oil, lard, beef bone fat, Japan wax kernel oil, hydrogenated oil, neatsfoot oil, Japan wax, and hydrogenated castor oil.

Examples of hydrocarbon oils include liquid paraffin, ozokerite, squalane, pristine, paraffin, ceresin, squalene, petrolatum, and microcrystalline wax.

Examples of silicone oils include chained polysiloxane (e.g. dimethylpolysiloxane, methylphenylpolysiloxane, and diphenylpolysiloxane); cyclic polysiloxane (e.g. octamethylcyclotetrasiloxane, decamethylcyclopentasiloxane, and dodecamethylcyclohexasiloxane); silicone resin having a three-dimensional network structure; silicone rubber, a variety of modified polysiloxane (e.g. amino modified polysiloxane, polyether modified polysiloxane, alkyl modified polysiloxane, polyether/alkyl comodified polysiloxane, fluorine modified polysiloxane, polyoxyethylene/polyoxypropylene copolymer modified polysiloxane, linear amino polyether modified polysiloxane, amidoalkyl modified polysiloxane, aminoglycol modified polysiloxane, aminophenyl modified polysiloxane, carbinol modified polysiloxane, polyglycerin modified polysiloxane, and polyglycerin/alkyl comodified polysiloxane); dimethiconol; and acrylic silicone. As a mixing condition, silicone oils may be solubilized or emulsified in a composition. Also, a particle when emulsified is the same size as in the case of a general cleansing composition.

Examples of cationic surfactants include alkyltrimethylammonium salts (e.g. stearyl trimethyl ammonium chloride, lauryl trimethyl ammonium chloride, and behenyl trimethyl ammonium chloride); alkyl pyridinium salts (e.g. cetylpyridinium chloride); distearyl dimethyl ammonium chloride; dialkyl dimethyl ammonium salt; poly (N,N′-dimethyl-3,5-methylenepiperidinium) chloride; alkyl quaternary ammonium salts; alkyl dimethyl benzyl ammonium salts; alkyl isoquinolinium salts; dialkyl morphonium salts; POE alkyl amines; alkyl amine salts; polyamine fatty acid derivatives; amyl alcohol fatty acid derivatives; benzalkonium chloride; and benzethonium chloride.

Examples of nonionic surfactants include fatty acid alkanolamides such as coconut oil fatty acid monoethanolamide, coconut oil fatty acid diethanolamide, lauric acid isopropanolamide, and oleic acid diethanolamide; sorbitan fatty acid esters such as sorbitan monostearate, and sorbitan sesquioleate; alkylene glycol fatty acid esters such as diethylene glycol laurate, propylene glycol laurate, ethylene glycol monooleate, and ethylene glycol distearate; hydrogenated castor oil derivatives; glycerin alkyl ethers; POE sorbitan fatty acid esters such a as POE sorbitan monooleate, and POE sorbitan monostearate; POE sorbitol fatty acid esters such as POE sorbitol monolaurate; POE glycerin fatty acid esters such as POE glycerin monoisostearate; polyethylene glycol monooleate; POE glycerin fatty acid esters such as POE distearate; POE alkyl ethers such as POE octyldodecyl ether; POE alkyl phenyl ethers such as POE nonyl phenyl ether; POE/POP alkyl ethers; Pluronic types; POE castor oil; POE hydrogenated castor oil derivatives; sugars such as sugar esters, sugar ethers, and sugar amides; and alkylglycosides.

Examples of powders constituent include inorganic powder (e.g. talc, kaolin, mica, sericite, muscovite, phlogopite, synthetic mica, lepidolite, biotite, vermiculite, magnesium carbonate, calcium carbonate, aluminium silicate, barium silicate, calcium silicate, magnesium silicate, strontium silicate, tungsten acid metal salt, magnesium, silica, zeolite, barium sulfate, calcium sulfate [burnt plaster], calcium phosphate, fluoroapatite, hydroxyapatite, ceramic powder, metallic soaps [e.g. zinc myristate, calcium palmitate, and aluminium stearate]), and boron nitride; organic powder (e.g. polyamide resin powder [nylon powder], polyethylene powder, polymethylmethacrylate powder, polystyrene powder, styrene/acrylic acid copolymer resin powder, benzoguanamine resin powder, polytetrafluoroethylene powder, and cellulose powder); inorganic white pigment (e.g. titanium dioxide, and zinc oxide); inorganic red pigment (e.g. iron oxide [Bengala], and iron titanate); inorganic brown pigment (e.g. gamma-iron oxide); inorganic yellow pigment (e.g. yellow iron oxide, and yellow ocher); inorganic black pigment (e.g. black iron oxide, and lower titanium oxide); inorganic violet pigment (e.g. mango violet, and cobalt violet); inorganic green pigment (e.g. chromium oxide, chromium hydroxide, and cobalt titanate); inorganic blue pigment (e.g. ultramarine, and Prussian blue); pearl pigment (e.g. titanium oxide coated mica, titanium oxide coated bismuth oxychloride, titanium oxide coated talc, colored titanium oxide coated mica, bismuth oxychlorid, and argentine); metallic powder pigment (e.g. aluminum powder, and copper powder); zirconium, barium or aluminum lake organic pigments (e.g. organic pigment such as Red No. 201, Red No. 202, Red No. 204, Red No. 205, Red No. 220, Red No. 226, Red No. 228, Red No. 405, Orange No. 203, Orange No. 204, Yellow No. 205, Yellow No. 401 and Blue No. 404; Red No. 3, Red No. 104, Red No. 106, Red No. 227, Red No. 230, Red No. 401, Red No. 505, Orange No. 205, Yellow No. 4, Yellow No. 5, Yellow No. 202, Yellow No. 203, Green No. 3 and Blue No. 1); natural pigment (e.g. chlorophyll, and beta-carotene); and clay mineral (e.g. bentonite, hectorite, and laponite).

Examples of moisturizers include polyethylene glycol, propylene glycol, isoprene glycol, glycerin, 1,3-butylene glycol, xylitol, sorbitol, maltitol, chondroitin sulfate, hyaluronan, mucoitinsulfuric acid, caronic acid, atelocollagen, cholesteryl-12-hydroxystearate, sodium lactate, bile salts, dl-pyrrolidone carboxylate, short-chain soluble collagens, diglycerin (EO)PO adducts, Rosa roxburghii extract, yarrow extract, and melilot extract.

Examples of natural water-soluble polymers include plant-derived polymers (e.g. gum arabic, tragacanth gum, galactan, guar gum, carob gum, karaya gum, carrageenan, tamarind gum, locust bean gum, pectin, agar, quince seed [marmelo], algal colloid (brown alga extract), starch [rice, corn, potatoe, wheat], and glycyrrhizinic acid); microorganism-derived polymers (e.g. xanthan gum, dextran, succinoglucan, and pullulan); and animal-derived polymers (e.g. collagen, casein, albumin, and gelatin). Also, their derivatives (POP/POE modified, alkyl modified, cationized, anionized or silylated derivatives) may be included.

Examples of semisynthetic water-soluble polymers include, starch polymers (e.g. carboxymethyl starch, and methyl hydroxypropyl starch); cellulose polymers (e.g. methyl cellulose, ethyl cellulose, methylhydroxypropyl cellulose, hydroxyethyl cellulose, sodium cellulose sulfate, dialkyldimethylammonium sulfate cellulose, hydroxypropyl cellulose, carboxymethyl cellulose, sodium carboxymethyl cellulose, crystalline cellulose, cellulose powder, hydrophobically modified compounds of these polymers [e.g. partially stearoxy modified compounds], and cation modified compounds of these polymers); alginate polymers (e.g. sodium slginate, and propylene glycol alginate); and sodium pectate.

Examples of synthetic water-soluble polymers include vinyl polymers (e.g. polyvinyl alcohol, polyvinyl methyl ether, polyvinylpyrrolidone, and carboxyvinyl polymer); polyoxyethylene polymers (e.g. polyoxyethylene/polyoxypropylene copolymers, for example, polyethylene glycol 20,000, 40,000 or 60,000); poly(dimethyldiallylammonium halide) type cationic polymers (e.g. Merquat 100 manufactured by Merck & Co., Inc.); dimethyldiallylammonium halide/acrylamido copolymer type cationic polymers (e.g. Merquat 550 manufactured by Merk & Co., Inc.); acrylic polymers (e.g. sodium polyacrylate, polyethyl acrylate, and polyacrylamide); polyethyleneimine; cationic polymers; magnesium aluminum silicate (veegum); and polyquaternium-39.

Examples of ultraviolet absorbers include benzoic acid UV absorbers (e.g. p-aminobenzoic acid [hereinafter abbreviated as PABA], PABA monoglycerine ester, N,N-dipropoxy PABA ethyl ester, N,N-diethoxy PABA ethyl ester, N,N-dimethyl PABA ethyl ester, and N,N-dimethyl PABA butyl ester); anthranilic acid UV absorbers (e.g. homomethyl N-acetylanthranilate); salicylic acid UV absorbers (e.g. amyl salicylate, methyl salicylate, homomethyl salicylate, octyl salicylate, phenyl salicylate, benzyl salicylate, and p-isopropanolphenyl salicylate); cinnamic acid UV absorbers (e.g. octyl cinnamate, ethyl 4-isopropylcinnamate, methyl 2,5-diisopropylcinnamate, ethyl 2,4-diisopropylcinnamate, methyl 2,4-diisopropylcinnamate, propyl p-methoxycinnamate, isopropyl p-methoxycinnamate, isoamyl p-methoxycinnamate, octyl p-methoxycinnamate [2-ethylhexyl p-methoxycinnamate], 2-ethoxyethyl p-methoxycinnamate, cyclohexyl p-methoxycinnamate, ethyl α-cyano-β-phenylcinnamate, 2-ethylhexyl-α-cyano-β-phenylcinnamate, and glyceryl mono-2-ethylhexanoyl-diparamethoxy cinnamate); benzophenone UV absorbers (e.g. 2,4-dihydroxybenzophenone, 2,2′-dihydroxy-4-methoxybenzophenone, 2,2′-dihydroxy-4,4′-dimethoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl-4′-phenyl-benzophenone-2-carboxylate, 2-hydroxy-4-n-octoxybenzophenone, and 4-hydroxy-3-carboxybenzophenone); 3-(4′-methylbenzylidene)-d,l-camphor and 3-benzylidene-d.l-camphor; 2-phenyl-5-methylbenzoxazol; 2,2′-hydroxy-5-methylphenylbenzotriazol; 2-(2′-hydroxy-5′-t-octylphenyl) benzotriazol; 2-(2′-hydroxy-5′-methylphenylbenzotriazol; dianisoylmethane; 4-methoxy-4′-t-butyldibenzoylmethane; 5-(3,3-dimethyl-2-norbornylidene)-3-pentane-2-one; and triazine UV absorbers (e.g. 2-{4 [(2-hydroxy-3-dodecyloxypropyl)oxy]-2-hydroxyphenyl}-4,6-bis(2,4-dimethylphenyl)-1,3,5-triazine, and 2-{4 [(2-hydroxy-3-tridecyloxypropyl)oxy]-2-hydroxyphenyl}-4,6-bis(2,4-dimethylphenyl)-1,3,5-triazine).

Examples of sequestering agents include 1-hydroxyethane-1,1-diphosphonic acid, 1-hydroxyethane-1,1-diphosphonic acid tetrasodium salt, disodium edetate, trisodium edetate, tetrasodium edetate, sodium citrate, sodium polyphosphate, sodium metaphosphate, gluconic acid, phosphoric acid, citric acid, ascorbic acid, succinic acid, edetic acid, and trisodium hydroxyethyl ethylenediamine triacetate.

Examples of pH adjusters include buffers such as lactic acid/sodium lactate, citric acid/sodium citrate, and succinic acid/sodium succinate.

Examples of vitamins include vitamins A, B1, B2, B6, C and E and the derivatives thereof, panthothenic acid and the derivatives thereof, and biotin.

Examples of antioxidants include tocopherols, dibutylhydroxytoluene, butylhydroxyanisole, and gallic acid esters.

Examples of other components which can be contained include antiseptic (such as ethylparaben, butylparaben, 1,2-alkane diol [the carbon chain length of C6 to C14] and the derivatives thereof, phenoxyethanol, and methylchloroisothiazolinone); antiphlogistic (such as glycyrrhizic acid derivatives, glycyrrhetinic acid derivatives, salicylic acid derivatives, hinokitiol, zinc oxide, and allantoin); whitening agent (such as saxifrage sarmentosa extract and arbutin); various extracts (such as phellodendron bark, goldthread, lithospermum root, paeonia albiflora, swertiajaponica, birch, sage, loquat, carrot, aloe, malya sylvestris [mallow], iris, vitis vinifera [grape], coix lacryma-jobi [job's tears], luffa cylindrica, lily, saffron, cnidium officinale, ginger, hypericum perforatum, ononis spinosa, allium sativum [gerlic], capsicum frutescens, citrus unshiu peel, angelica acutiloba, and sea alga); activator agent (such as royal jelly, photosensitizers, and cholesterol derivatives); blood circulation accelerator (such as nonylic acid vanillylamide, nicotinic acid benzyl esters, nicotinic acid β-butoxy ethyl esters, capsaicin, Zingerone, Cantharides tincture, ichthammol, tannic acid, α-borneol, tocopherol nicotinate, inositol hexanicotinate, cyclandelate, cinnarizine, tolazoline, acetylcholine, verapamil, cepharanthine, and γ-orizanol); antiseborrheic agent (such as sulfur and thianthol); and anti-inflammatory agent (such as tranexamic acid, thiotaurine, and hypotaurine); and aromatic alcohols (such as benzyl alcohol and benzyloxy ethanol).

When the shampoo composition according to the present invention is applied to the hair, the quaternary ammonium group-containing silylated urethane polymer quickly adsorbs to the hair surface strongly, and it will not be washed away even by rinsing with water. Therefore, the penetration of water into the inside of the hair when it is washed can be inhibited; the outflow of a dye from the inside of the hair can be inhibited; and the excellent fading-inhibiting effect can be exhibited.

Further, the fading-inhibiting effect is further accelerated by a combined use of a specific anionic surfactant, amphoteric surfactant, and cationic conditioning polymer, and good use feeling such as finger-running-through-hair properties and pliability during rinsing can be imparted to the hair after dyeing.

Therefore, when a shampoo or the like including the shampoo composition according to the present invention is used for daily shampoo use, the fading of hair color can be significantly inhibited, and beautiful hair color can be maintained. At the same time, the stiffness and the like due to dyeing can be relieved to obtain pliable hair having good finger-running-through-hair properties.

Note that the color of the hair before and after applying shampoo or the like to the hair to which a hair coloring is applied is measured using a spectral colorimeter, and the fading-inhibiting effect of hair color by the shampoo composition according to the present invention can be evaluated by the color difference (ΔE) of the hair. A color difference (ΔE) closer to zero means higher fading-inhibiting effect.

When applying shampoo treatment (treatment of repeating washing-rinsing-drying 5 times) using the shampoo composition according to the present invention, the color difference (ΔEs) of the hair before and after the shampoo treatment is for example 2.05 or less, preferably 2.00 or less, and particularly preferably 1.60 or less. If the color difference exceeds the above range, it tends to be difficult to realize a fading-inhibiting effect.

### EXAMPLES

Hereinafter, the present invention will be more specifically described with reference to Examples, but the present invention is not limited to these Examples. Note that, in the following description, unless otherwise specified, “part” refers to “part by mass”, and “%” refers to “mass %”.

First, the evaluation methods of the fading-inhibiting effect and the use feeling used in the following tests will be described.

### <Evaluation Method of Fading-Inhibiting Effect>

1. A bundle of 100% white hair (manufactured by Beaulax Co., Ltd.) was dyed using a brown hair coloring (trade name “Dianist NB8”, manufactured by Shiseido Professional Inc.).
2. The hair color of the hair bundle which is subjected to dyeing (dyed hair bundle) was measured using a spectral colorimeter (trade name “CM-2500d”, manufactured by Konica Minolta Co., Ltd.) (C₁).
3. The dyed hair bundle was subjected to washing treatment (treatment of repeating washing-rinsing-drying 5 times) using a shampoo obtained in Examples and Comparative Examples (the sample number was set to 10 for each shampoo).
4. The hair color of the dyed hair bundle after washing was measured using a spectral colorimeter in the same manner as described above; the average value (C₂) of the measured hair color was calculated; the color difference (ΔEs: C₁-C₂) before and after the washing treatment was determined; the color difference of each Example was compared with that of Comparative Example; and the fading-inhibiting effect was evaluated in accordance with the following evaluation criteria.

Evaluation Criteria

The value of (ΔEs of each Example)-(ΔEs of Comparative Example) is −1 or less: \hat{∘}\hat{∘}

The value of (ΔEs of each Example)-(ΔEs of Comparative Example) exceeds −1 and −0.5 or less: \hat{∘}

The value of (ΔEs of each Example)-(ΔEs of Comparative Example) exceeds −0.5 and 0 or less: ◯

The value of (ΔEs of each Example)-(ΔEs of Comparative Example) exceeds 0: X

### <Evaluation Method of Use Feeling>

A sensory test was carried out by 10 professional panelists on the dyed hair bundle after washing, and the finger-running-through-hair properties and pliability of the hair during rinsing in Examples as compared with those in Comparative Examples were evaluated in accordance with the following evaluation criteria.

70% or more of the panelists answered that Examples were equivalent or good as compared with Comparative Examples: \hat{∘}\hat{∘}

50% or more and less than 70% of the panelists answered that Examples were equivalent or good as compared with Comparative Examples: \hat{∘}

30% or more and less than 50% of the panelists answered that Examples were equivalent or good as compared with Comparative Examples: ◯

Less than 30% of the panelists answered that Examples were equivalent or good as compared with Comparative Examples: X

Next, a method for producing a quaternary ammonium group-containing urethane polymer used in the following tests will be described.

### Preparation Example 1 of Quaternary Ammonium Group-Containing Silylated Urethane Polymer

### Preparation Example of Ester-Modified Amino Group-Containing Alkoxysilane (Compound A)

γ-Aminopropyl triethoxysilane (trade name “KBE903”, manufactured by Shin-Etsu Chemical Co., Ltd.) in an amount of 221.4 parts was mixed with 240.4 parts of lauryl acrylate, and the mixture was allowed to react with each other at 50° C. for 7 days to obtain an ester-modified amino group-containing alkoxysilane (compound A).

### Production Example 1

In a four-necked flask equipped with a nitrogen introducing tube, a thermometer, a condenser, and a stirrer, were blended 50 parts of polytetramethylene ether glycol (number average molecular weight: 1944.5, trade name “PTMG 2000”, manufactured by Mitsubishi Chemical Corporation), 25 parts of trimethylolpropane mono(polyethylene oxide methyl ether) (number average molecular weight: 1089.3, trade name “Ymer N120”, manufactured by Perstorp Inc.) represented by the following formula (2), 25 parts of N-methyl-N,N-diethanolamine (MDA), 60.4 parts of isophorone diisocyanate (IPDI), 50 parts of methyl ethyl ketone (MEK), and 0.1 part of 1,8-diazabicyclo[5.4.0]undec-7-ene (DBU) as a catalyst, and the mixture was allowed to react with each other at a temperature of 80 to 85° C. in a nitrogen atmosphere for 5 hours to obtain a reaction mixture containing a tertiary amine-containing urethane polymer.

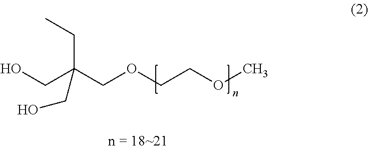

To the reaction mixture containing the urethane polymer, was added 150 parts of methyl ethyl ketone (MEK) followed by cooling the mixture to 50° C., and thereto was added 25.2 parts of dimethyl sulfate as a quaternizing agent to convert the tertiary amine part to a quaternary ammonium ion over 30 minutes to 1 hour at 50 to 60° C. to produce a quaternary ammonium group-containing urethane polymer.

The reaction mixture of the quaternary ammonium group-containing urethane polymer was blended and mixed with 9.9 parts of the compound A obtained in Preparation Example 1, and the mixture was allowed to react with each other at a temperature of 65 to 75° C. for 1 hour in a nitrogen atmosphere to obtain a reaction mixture (1) containing a quaternary ammonium group-containing silylated urethane polymer.

Next, the reaction mixture (1) was cooled to 40° C., and then thereto was added 1000 parts of deionized water with high speed stirring. Subsequently, the solvent was distilled off at 45 to 50° C. under reduced pressure to obtain an aqueous dispersion (1).

The evaluations described above (fading-inhibiting effect and use feeling) were made on each shampoo composition produced by a recipe shown in the following Tables 1 and 2. The results are shown in Tables 1 and 2. Note that a comparison between Example and Comparative Example in each evaluation was made on the basis of Comparative Example 1-1.

TABLE 1

| | EXAMPLE | | | | | | | |
|---|---|---|---|---|---|---|---|---|
| | 1•1 | 1•2 | 1•3 | 1•4 | 1•5 | 1•6 | 1•7 | 1•8 |
| Coconut oil fatty acid methyltaurine | 3 | 6 | 10 | 12 | 15 | 6 | 20 | 3 |
| sodium salt | | | | | | | | |
| Sodium POE (2) lauryl ether sulfate | — | — | — | — | — | — | — | — |
| Coconut oil fatty acid amide propyl | 3 | 6 | 10 | 12 | 6 | 15 | 3 | 20 |
| betaine | | | | | | | | |
| Cationized cellulose | 0.5 | 0.5 | 0.5 | 0.5 | 0.5 | 0.5 | 0.5 | 0.5 |
| Quaternary ammonium group-containing | 0.2 | 0.2 | 0.2 | 0.2 | 0.2 | 0.2 | 0.2 | 0.2 |
| urethane polymer | | | | | | | | |
| Coconut oil fatty acid diethanolamide | 2 | 2 | 2 | 2 | 2 | 2 | 2 | 2 |
| Propylene glycol | 1 | 1 | 1 | 1 | 1 | 1 | 1 | 1 |
| Citric acid | 0.1 | 0.1 | 0.1 | 0.1 | 0.1 | 0.1 | 0.1 | 0.1 |
| Purified water | 90.2 | 84.2 | 76.2 | 72.2 | 75.2 | 75.2 | 73.2 | 73.2 |
| Δ E s | 1.0 | 0.9 | 1.1 | 1.2 | 1.5 | 1.3 | 1.8 | 1.5 |
| Fading-inhibiting Effect | ⊚⊚ | ⊚⊚ | ⊚⊚ | ⊚⊚ | ⊚ | ⊚⊚ | ⊚ | ⊚ |
| Use Feeling (finger-running-through-hair | ⊚ | ⊚ | ⊚ | ⊚ | ⊚ | ⊚ | ◯ | ◯ |
| properties during rinsing) | | | | | | | | |
| Use Feeling (pliability of the hair | ◯ | ◯ | ◯ | ◯ | ◯ | ◯ | ◯ | ◯ |
| during rinsing) | | | | | | | | |

TABLE 2

| | COMPARATIVE EXAMPLE | | | |
|---|---|---|---|---|
| | 1-1 | 1-2 | 1-3 | 1-4 |
| Coconut oil fatty acid methyltaurine | — | — | 0.5 | — |
| sodium salt | | | | |
| Sodium POE (2) lauryl ether sulfate | 14 | 14 | 14 | 14 |
| Coconut oil fatty acid amide propyl | — | — | — | 0.5 |
| betaine | | | | |
| Cationized cellulose | 0.5 | 0.5 | 0.5 | 0.5 |
| Quaternary ammonium group-containing | — | 0.2 | 0.2 | 0.2 |
| urethane polymer | | | | |
| Coconut oil fatty acid diethanolamide | 2 | 2 | 2 | 2 |
| Propylene glycol | 1 | 1 | 1 | 1 |
| Citric acid | 0.1 | 0.1 | 0.1 | 0.1 |
| Purified water | 82.4 | 82.2 | 81.7 | 81.7 |
| ΔEs | 2.3 | 1.4 | 1.4 | 1.4 |
| Fading-inhibiting Effect | — | ⊚ | ⊚ | ⊚ |
| Use Feeling (finger-running-through-hair | — | X | X | X |
| properties during rinsing) | | | | |
| Use Feeling (pliability of the hair during | — | X | X | X |
| rinsing) | | | | |

### (Production Method)

Propylene glycol and canonized cellulose were added to purified water and sufficiently dissolved with stirring, and then thereto were successively added remaining components to obtain a shampoo composition.

The fading-inhibiting effect of Comparative Example 1-2 was higher than that of Comparative Example 1-1 in Tables 1 and 2. Therefore, it is obvious that the fading-inhibiting effect is improved by blending a quaternary ammonium group-containing urethane polymer.

Further, Examples 1-1 to 1-8 in which both a taurine-derivative surfactant (coconut oil fatty acid methyltaurine sodium salt) which is an anionic surfactant and an alkylamide betaine amphoteric surfactant (coconut oil fatty acid amide propyl betaine) were blended showed improvement in finger-running-through-hair properties and pliability during rinsing as compared with Comparative Examples 1-1 and 1-2 in which an alkyl ether sulfate (sodium POE(2) lauryl ether sulfate) which is an anionic surfactant was used as a cleaning agent, Comparative Example 1-3 in which a taurine-derivative surfactant and sodium POE (2) lauryl ether sulfate were blended, and Comparative Example 1-4 in which an alkylamide betaine amphoteric surfactant and sodium POE (2) lauryl ether sulfate were blended.

Although the above effect was sufficiently observed also in Example 1-7 in which the amount of the taurine-derivative surfactant was set to 20 mass %, the fading-inhibiting effect and use feeling were particularly remarkably improved in Examples 1-1 to 1-4 in which the taurine-derivative surfactant was blended in an amount of 3 to 12 mass %. Note that when studied more in detail, the blending effect of the taurine-derivative surfactant was observed when it was blended in an amount of 1 mass % or more.

Further, as shown by the results of Example 1-6 relative to those of Example 1-2, a good effect is maintained even if the blending of an alkylamide betaine amphoteric surfactant is increased, but when the results of Example 1-8 relative to those of Example 1-1 are taken into consideration, it is considered to be suitable to set the amount of the alkylamide betaine amphoteric surfactant blended to about 20 mass % or less. Note that when studied more in detail, the blending effect of the alkylamide betaine amphoteric surfactant was sufficiently observed when it was blended in an amount of 1 mass % or more.

Therefore, in the present invention, it is preferred to blend the taurine-derivative surfactant as an anionic surfactant and the alkylamide betaine surfactant as an amphoteric surfactant in combination with a quaternary ammonium group-containing urethane polymer, and the amount of the taurine-derivative surfactant blended is preferably 1 to 20 mass %, more preferably 3 to 12 mass %. Further, it is suitable to set the amount of the alkylamide betaine surfactant blended to 1 to 20 mass %.

Further, the evaluations described above (use feeling) were made on each shampoo composition produced by a recipe shown in the following Tables 3 and 4. The results are shown in Tables 3 and 4. Note that a comparison between Example and Comparative Example in each evaluation was made on the basis of Comparative Example 2-1.

TABLE 3

| | EXAMPLE | | | | | | | | | |
|---|---|---|---|---|---|---|---|---|---|---|
| | 2•1 | 2•2 | 2•3 | 2•4 | 2•5 | 2•6 | 2•7 | 2•8 | 2•9 | 2•10 |
| Coconut oil fatty acid methyltaurine | 6 | 6 | 6 | 6 | 6 | 6 | 6 | 6 | 6 | 6 |
| sodium salt | | | | | | | | | | |
| Coconut oil fatty acid amide propyl | 6 | 6 | 6 | 6 | 6 | 6 | 6 | 6 | 6 | 6 |
| betaine | | | | | | | | | | |
| Trimethylaminopropylacrylamide chloride/ | 0.02 | 0.1 | 0.2 | 0.5 | 1 | 2 | — | — | — | — |
| dimethylacrylamide copolymer (*1) | | | | | | | | | | |
| Acrylic acid/methyl acrylate/methacrylamide | — | — | — | — | — | — | 0.02 | 0.1 | 0.2 | 0.5 |
| propyltrimethylammonium chloride | | | | | | | | | | |
| copolymer (*2) | | | | | | | | | | |
| Cationized guar gum | — | — | — | — | — | — | — | — | — | — |
| Quaternary ammonium group-containing | 0.2 | 0.2 | 0.2 | 0.2 | 0.2 | 0.2 | 0.2 | 0.2 | 0.2 | 0.2 |
| urethane polymer | | | | | | | | | | |
| Coconut oil fatty acid diethanolamide | 2 | 2 | 2 | 2 | 2 | 2 | 2 | 2 | 2 | 2 |
| Propylene glycol | 1 | 1 | 1 | 1 | 1 | 1 | 1 | 1 | 1 | 1 |
| Citric acid | 0.1 | 0.1 | 0.1 | 0.1 | 0.1 | 0.1 | 0.1 | 0.1 | 0.1 | 0.1 |
| Purified water | 84.68 | 84.6 | 84.5 | 84.2 | 83.7 | 82.7 | 84.68 | 84.6 | 84.5 | 84.2 |
| Use Feeling (finger-running-through-hair | ⊚⊚ | ⊚⊚ | ⊚⊚ | ⊚⊚ | ⊚⊚ | ⊚ | ⊚⊚ | ⊚⊚ | ⊚⊚ | ⊚⊚ |
| properties during rinsing) | | | | | | | | | | |
| Use Feeling (pliability of the hair | ⊚⊚ | ⊚⊚ | ⊚⊚ | ⊚⊚ | ⊚ | ⊚ | ⊚⊚ | ⊚⊚ | ⊚⊚ | ⊚⊚ |
| during rinsing) | | | | | | | | | | |

TABLE 4

| | | | | | COMPARATIVE |
|---|---|---|---|---|---|
| | EXAMPLE | | | | EXAMPLE |
| | 2-11 | 2-12 | 2-13 | 2-14 | 2-1 |
| Coconut oil fatty acid methyltaurine sodium salt | 6 | 6 | 6 | 6 | 6 |
| Coconut oil fatty acid amide propyl betaine | 6 | 6 | 6 | 6 | 6 |
| Trimethylaminopropylacrylamide chloride/dimethylacrylamide copolymer (*1) | — | — | 0.01 | 0.5 | — |
| Acrylic acid/methyl acrylate/methacrylamide propyltrimethylammonium chloride copolymer (*2) | 1 | 2 | 0.01 | 0.5 | — |
| Cationized guar gum | — | — | — | — | 0.5 |
| Quaternary ammonium group-containing urethane polymer | 0.2 | 0.2 | 0.2 | 0.2 | 0.2 |
| Coconut oil fatty acid diethanolamide | 2 | 2 | 2 | 2 | 2 |
| Propylene glycol | 1 | 1 | 1 | 1 | 1 |
| Citric acid | 0.1 | 0.1 | 0.1 | 0.1 | 0.1 |
| Purified water | 83.7 | 82.7 | 84.68 | 83.7 | 84.2 |
| Use Feeling (finger-running-through-hair properties during rinsing) | ⊚⊚ | ⊚ | ⊚⊚ | ⊚⊚ | — |
| Use Feeling (pliability of the hair during rinsing) | ⊚ | ◯ | ⊚⊚ | ⊚ | — |

(*1) DIASLEEK C-822 (manufactured by Mitsubishi Chemical Corporation)
(*2) Merquat 2001 (manufactured by Nalco Japan, Co., Ltd.)

### (Production Method)

Cationic conditioning polymer was added to purified water and sufficiently dissolved with stirring, and then thereto were successively added remaining components to obtain a shampoo composition.

As shown in Tables 3 and 4, Examples in which a trimethylaminopropylacrylamide chloride/dimethylacrylamide copolymer or an acrylic acid/methyl acrylate/methacrylamide propyltrimethylammonium chloride copolymer having a MAPTAC structure, or both of the copolymers are used as a cationic conditioning polymer showed good use feeling as compared with Comparative Example 2-1 in which cationized guar gum was used as the cationic conditioning polymer.

Further, in the comparison between Examples, a tendency of reduction in the use feeling was observed when the amount blended was excessively high, in any case where any cationic conditioning polymer was used. According to the results in Table 3, the blending of the polymer in an amount of 0.02 to 1 mass % was particularly suitable, but as a result of more detailed study, it was found that the use feeling was sufficiently improved by blending the polymer in an amount of 0.01 to 2 mass %.

Therefore, in the present invention, it is preferred to use a cationic polymer having a MAPTAC structure as a cationic conditioning polymer, and the amount thereof blended is preferably 0.01 to 2 mass %, more preferably 0.02 to 1 mass %.

The evaluations described above (fading-inhibiting effect and use feeling) were made on each shampoo composition produced by a recipe shown in the following Table 5. The results are shown in Table 5. Note that a comparison between Example and Comparative Example in each evaluation was made on the basis of Comparative Example 3-1.

TABLE 5

| | | | | | | | COMPARATIVE | |
|---|---|---|---|---|---|---|---|---|
| | EXAMPLE | | | | | | EXAMPLE | |
| | 3•1 | 3•2 | 3•3 | 3•4 | 3•5 | 3•6 | 3•1 | 3•2 |
| Coconut oil fatty acid methyltaurine | 6 | 6 | 6 | 6 | 6 | 6 | 6 | 6 |
| sodium salt | | | | | | | | |
| Coconut oil fatty acid amide propyl | 6 | 6 | 6 | 6 | 6 | 6 | 6 | 6 |
| betaine | | | | | | | | |
| Trimethylaminopropylacrylamide chloride/ | 0.5 | 0.5 | 0.5 | 0.5 | 0.5 | 0.5 | 0.5 | 0.5 |
| dimethylacrylamide copolymer (*1) | | | | | | | | |
| Quaternary ammonium group-containing | 0.01 | 0.05 | 0.1 | 0.2 | 0.6 | 1 | — | 1.1 |
| urethane polymer | | | | | | | | |
| Coconut oil fatty acid diethanolamide | 2 | 2 | 2 | 2 | 2 | 2 | 2 | 2 |
| Propylene glycol | 1 | 1 | 1 | 1 | 1 | 1 | 1 | 1 |
| Citric acid | 0.1 | 0.1 | 0.1 | 0.1 | 0.1 | 0.1 | 0.1 | 0.1 |
| Purified water | 84.39 | 84.35 | 84.3 | 84.2 | 83.8 | 83.4 | 84.4 | 83.3 |
| Δ E s | 1.5 | 1.1 | 1.1 | 1.0 | 0.9 | 0.9 | 2.2 | 0.9 |
| Fading-inhibiting Effect | ⊚ | ⊚⊚ | ⊚⊚ | ⊚⊚ | ⊚⊚ | ⊚⊚ | — | ⊚⊚ |
| Use Feeling (finger-running-through-hair | ⊚⊚ | ⊚⊚ | ⊚⊚ | ⊚⊚ | ⊚⊚ | ⊚ | — | X |
| properties during rinsing) | | | | | | | | |
| Use Feeling (pliability of the hair | ⊚⊚ | ⊚⊚ | ⊚⊚ | ⊚⊚ | ⊚⊚ | ◯ | — | X |
| during rinsing) | | | | | | | | |

*1: DIASLEEK C-822 (manufactured by Mitsubishi Chemical Corporation)

### (Production Method)

Cationic conditioning polymer was added to purified water and sufficiently dissolved with stirring, and then thereto were successively added remaining components to obtain a shampoo composition.

As shown in Table 5, Examples in which a quaternary ammonium group-containing urethane polymer was blended in an amount of 0.01 to 1 mass % showed improvement not only in the fading-inhibiting effect but also in the use feeling, as compared with Comparative Example 3-1 in which the polymer was not blended. In particular, when the amount of the quaternary ammonium group-containing urethane polymer blended was 0.05 to 0.6 mass %, both the fading-inhibiting effect and the use feeling significantly increased.

On the other hand, in Comparative Example 3-2 in which 1.1 mass % of the quaternary ammonium group-containing urethane polymer was blended, the fading-inhibiting effect was improved, but almost no improvement in the use feeling was observed.

Therefore, the amount of the quaternary ammonium group-containing urethane polymer blended in the present invention is 0.01 to 1 mass %, preferably 0.05 to 0.6 mass %.

1. A shampoo composition comprising:
(i) an anionic surfactant which is a taurine-derivative surfactant;
(ii) an amphoteric surfactant which is an alkylamide betaine surfactant;
(iii) a cationic conditioning polymer; and
(iv) 0.01 to 1 mass % of a quaternary ammonium group-containing silylated urethane polymer.

2. The shampoo composition according to claim 1, wherein the (iii) cationic conditioning polymer comprises one or more selected from a trimethylaminopropylacrylamide chloride/dimethylacrylamide copolymer and an acrylic acid/methyl acrylate/methacrylamide propyltrimethylammonium chloride copolymer.

3. The shampoo composition according to claim 1, wherein an amount of the (i) anionic surfactant blended is 1 to 20 mass %, and an amount of the (ii) amphoteric surfactant blended is 1 to 20 mass %.

4. The shampoo composition according to claim 1, wherein an amount of the (iii) cationic conditioning polymer blended is 0.01 to 2 mass %.

5. The shampoo composition according to claim 2, wherein an amount of the (i) anionic surfactant blended is 1 to 20 mass %, and an amount of the (ii) amphoteric surfactant blended is 1 to 20 mass %.

6. The shampoo composition according to claim 2, wherein an amount of the (iii) cationic conditioning polymer blended is 0.01 to 2 mass %.

7. The shampoo composition according to claim 3, wherein an amount of the (iii) cationic conditioning polymer blended is 0.01 to 2 mass %.
